(19) United States
(12) Patent Application Publication
(10) Pub. No.: US 2023/0329802 A1
(43) Pub. Date: Oct. 19, 2023

(54) ORGAN CONFORMABLE PANEL

(71) Applicant: Method AI, Inc., Cleveland, OH (US)

(72) Inventors: Jad Kaouk, Hunting Valley, OH (US); Charles D. Emery, Gilbert, AZ (US); Douglas Teany, Medfield, MA (US); John-Michael Sungur, Brookline, MA (US); Christopher Lee Laine, Bellingham, MA (US)

(73) Assignee: Method AI, Inc., Cleveland, OH (US)

(21) Appl. No.: 18/136,274

(22) Filed: Apr. 18, 2023

Related U.S. Application Data
(60) Provisional application No. 63/332,662, filed on Apr. 19, 2022.

(51) Int. Cl. A61B 8/00

(52) U.S. Cl. CPC A61B 8/4444; A61B 8/4483

(57) ABSTRACT

An ultrasound sensing system may include an organ conformable panel to be positioned on an exterior surface of an internal organ and a passive ultrasound detectable marker supported by the organ conformable panel. The organ conformable panel has a first acoustic impedance while the marker has a second acoustic impedance different than the first acoustic impedance.

### CROSS-REFERENCE TO RELATED PATENT APPLICATIONS

This application claims the benefit of U.S. Application No. 63/332,662 entitled ORGAN CONFORMABLE PANEL filed on Apr. 19, 2022 which is incorporated herein by reference in its entirety.

### BACKGROUND

Medical procedures, such as surgeries, sometimes involve the positioning and manipulation of a tool or effector within a patient. Precise and accurate positioning of the tool often presents a challenge. During surgery, portions of the organ may move, demanding real-time tool positioning and operation adjustments.

### BRIEF DESCRIPTION OF THE DRAWINGS

FIG. 1 is a diagram schematically illustrating portions of an example ultrasound sensing system interacting with an example organ.

FIG. 2 is a top view of an example organ conformable panel and passive ultrasound detectable marker of the example ultrasound sensing system of FIG. 1 positioned on the organ.

FIG. 3 is a top view of an example organ conformable panel and passive ultrasound detectable marker.

FIG. 4 is a sectional view of the example organ conformable panel and passive ultrasound detectable marker of FIG. 3 taken along line 4-4.

FIG. 5 is a sectional view of an example organ conformable panel and passive ultrasound detectable marker.

FIG. 6 is a sectional view of an example organ conformable panel and passive ultrasound detectable marker.

FIG. 7 is a top view of an example organ conformable panel supporting an example optical marker.

FIG. 8 is a bottom view of the example organ conformable panel of FIG. 7 supporting an example passive ultrasound detectable marker.

FIG. 9 is a sectional view of an example organ conformable panel, the example passive ultrasound detectable marker in the example optical marker of FIGS. 7 and 8 taken along line 9-9 of FIG. 7.

FIG. 10 is a sectional view of an example organ conformable panel supporting an example passive ultrasound detectable marker and an example optical marker taken along line 10-10 of FIG. 7.

FIG. 11 is a sectional view of an example organ conformable panel supporting an example passive ultrasound detectable marker.

FIG. 12 is a diagram schematically illustrating portions of an example ultrasound sensing system interacting with an example organ.

FIG. 13 is a top view of an example organ conformable panel and passive ultrasound detectable marker of the example ultrasound sensing system of FIG. 12 positioned on the organ.

FIG. 14 is a diagram schematically illustrating portions of an example ultrasound sensing system interacting with an example organ.

FIG. 15 is a top view of an example organ conformable panel and passive ultrasound detectable marker of the example ultrasound sensing system of FIG. 14 positioned on the organ.

FIG. 16 is a diagram schematically illustrating portions of an example ultrasound sensing system interacting with an example organ.

FIG. 17 is a top view of an example organ conformable panel and passive ultrasound detectable marker of the example ultrasound sensing system of FIG. 16 positioned on the organ.

FIG. 18 is a sectional view of the example organ conformable panel and passive ultrasound detectable marker of FIG. 17 taken along line 18-18.

FIG. 19 is a top view of an example organ conformable panel and passive ultrasound detectable marker of the example ultrasound sensing system of FIG. 16 positioned on the organ.

FIG. 20 is a sectional view of the example organ conformable panel and passive ultrasound detectable marker of FIG. 17 taken along line 20-20.

FIG. 21 is a sectional view of the example organ conformable panel and passive ultrasound detectable marker of FIG. 17 taken along line 21-21.

FIG. 22 is a top view of an example organ conformable panel and supporting example passive ultrasound detectable markers and positioned on the organ.

FIG. 23 is a bottom view of the example organ conformable panel of FIG. 22 supporting example optical markers and positioned on the organ.

FIG. 24 is a sectional view of the example organ conformable panel, passive ultrasound detectable markers and example optical markers of FIGS. 22 and 23 taken along line 24-24.

FIG. 25 is a sectional view of an example organ conformable panel, passive ultrasound detectable markers and example optical markers taken along line 25, 25 of FIG. 22.

FIG. 26 is a sectional view of an example organ conformable panel and a column of example passive ultrasound detectable markers for positioning on a surface of an organ.

FIG. 27 is a top view of portions of the example organ conformable panel and the column of example passive ultrasound detectable markers of FIG. 26.

FIG. 28 is a top view of portions of an example organ conformable panel supporting a grid of passive ultrasound detectable markers on an organ.

FIG. 29 is a sectional view of the example organ conformable panel and grid of passive ultrasound detectable markers taken along line 29-29 of FIG. 28.

FIG. 30 is a sectional view of an example organ conformable panel supporting a grid of passive ultrasound detectable markers taken along line 30-30 of FIG. 28.

FIG. 31 is a sectional view of an example organ conformable panel supporting a grid of passive ultrasound detectable markers taken along line 31-31 of FIG. 28.

FIG. 32 is a sectional view of an example organ conformable panel supporting a grid of passive ultrasound detectable markers and a grid of optical markers.

FIG. 33 is a sectional view of an example organ conformable panel supporting a grid of embedded passive ultrasound detectable markers.

FIG. 34 is a flow diagram of an example method for tracking or monitoring movement of an organ surface during a medical procedure on in organ.

FIG. 35 is a flow diagram of an example method 2100 for removing a portion of an internal organ, such as a tumor.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed are example ultrasound sensing systems that facilitate real-time monitoring of an organ of a patient while a medical procedure is being performed on the organ. The example ultrasound sensing systems monitor the target tissue of an organ, such as a tumor, in relation to a surgical tool where the localization of the target tissue relative to the surgical tool may be accomplished using ultrasound data or ultrasound images and other sensed data to mathematically describe the position of the target tissue relative to the surgical tool. As a result, such example ultrasound sensing systems may facilitate more precise and accurate positioning of a tool within the patient relative to the target tissue, compensating for movement of the tool and/or organ, such as movement caused by interaction of the tool with the organ or movement resulting from involuntary respiration.

The example ultrasound sensing systems utilize an organ conformable panel that is to be positioned on an exterior surface of an internal organ. The organ conformable panel may be flexible, stretchable or deformable to move with movement of the organ. The organ conformable panel has an acoustic impedance (also referred to as echogenicity) different than that of tissue of the organ upon which the organ conformable panel is secured. The organ conformable panel serves as a passive ultrasound detectable panel which may be used to track movement of particular portions of the organ during surgery.

The example ultrasound sensing systems may employ an organ surface ultrasound probe which is positioned on the exterior surface of the organ such that a field of view of the organ surface ultrasound probe contains those portions of the organ upon which the organ conformable panel is positioned. In some implementations, the organ surface ultrasound probe is positioned on a portion of the exterior surface of the organ that lies generally opposite to those portions of the organ upon which the organ conformable panel is positioned. The organ surface ultrasound probe detects ultrasound echoes from the organ conformable panel to track movement of the organ.

In some implementations, the organ conformable panel is composed of multiple different materials having different acoustic impedances/different echogenicities. As a result, distinct portions of the organ conformable panel are distinguishable from one another in the captured ultrasound data, permitting a controller to discern between the different portions of the organ conformable panel and their different locations. The controller may track movement of the different portions of the organ conformable panel based upon relative changes in the ultrasound echoes received from the different portions.

In some implementations, the different materials having different acoustic impedances may be provided at predetermined locations or distances relative to one another or relative to peripheral portions of the organ conformable panel. The predetermined locations or distances of the different materials may assist the controller in distinguishing between distinct portions of the organ conformable panel and tracking movement of the different portions of the organ conformable panel. In some implementations, the organ conformable panel may be formed from a first material having a first acoustic impedance, whereas passive ultrasound detectable markers supported by the organ conformable panel have a second acoustic impedance different than the first acoustic impedance.

In some implementations, the markers are formed on a surface of the organ conformable panel, either a face of the organ conformable panel which is to abut the exterior surface of the organ or the opposite face of the organ conformable panel. In some implementations, the markers are affixed or adhered to such surfaces. In some implementations, the markers are coated upon such surfaces such as by spraying, painting, printing or other application methods. In some implementations, the markers are encapsulated or embedded within the organ conformable panel, being spaced from both the front face and the rear face of the organ conformable panel. In some implementations, the markers are formed as part of a single integral unitary body with organ conformable panel. In some implementations, the markers are co-molded into or with the organ conformable panel.

In some example implementations, the markers may be provided with a predefined shape, location or pattern to assist in mapping of the exterior organ surface over which organ conformable panel is positioned. For example, in some implementations, the markers may comprise a two-dimensional array or grid of distinct dots or points, wherein the individual dots or points have an acoustic impedance different than that of the adjacent portions of the organ conformable panel extending between the dots or points. In some implementations, the markers may comprise a two-dimensional array or grid of continuous lines of material or dashed lines of material, wherein the material has acoustic impedance distinct from the acoustic impedance of the remaining or adjacent portions of the organ conformable panel.

In particular example implementations, the passive ultrasound detectable markers supported by the organ conformable panel may be provided for alignment with a targeted portion of the underlying organ for which a surgical procedure is to be performed. For example, the markers may be in the form of a bull's-eye, an arrow, a circle, a square, a triangle, a star, or another shape which is to be positioned on the external surface of the organ based upon the location of the portion of the organ targeted for surgery, such as removal. During surgery, the marker or markers may be detected by an ultrasound probe, wherein the ultrasound data or ultrasound images generated by the ultrasound echoes from the marker or markers may be used to assist in the positioning of and/or control of the surgical tool. In some implementations, the markers may be used to identify those portions of the organ to be removed.

In some implementations, the markers may be used to identify those portions of the organ which are not to be removed or which may be sensitive to surgical errors or inaccuracies. For example, such markers may be utilized to identify those portions of an organ that, if accidentally severed or removed, would result in more severe consequences. In such implementations, a medical practitioner controlling the surgical tool or the robotic system controlling the surgical tool may automatically adjust operation or characteristics of the surgery when in or approaching such identified regions. For example, a medical practitioner controlling the surgical tool or a robotic system controlling the surgical tool may automatically reduce the speed at which surgical tool is cutting or is moving when approaching or within those regions identified by the markers and as detected by the ultrasound probe.

In some implementations, edges of the organ conformable panel itself may serve to identify those portions of the underlying organ which are to be removed or which are not to be removed during a surgery. In some implementations, the outer peripheral edge of the organ conformable panel may identify those portions of the organ targeted for removal. In such implementations, the size and shape of the organ conformable panel may generally match the size and shape of the portion of the exterior surface of the organ which is to be removed. In such an implementation, a cutting tool may be manipulated to cut the organ along the outer edge of the organ conformable panel, wherein the outer peripheral edge of the organ conformable panel is detected by the ultrasound probe during the procedure.

In some implementations, an inner peripheral edge of the organ conformable panel made identify those portions of the organ targeted for removal. In some implementations, organ conformable panel may include an aperture therethrough, wherein the aperture is positioned directly over the portion of the organ to be removed, such as a tumor. The size and shape of the aperture may correspond to the size and shape of the portion of the organ designated for removal. In such an implementation, a cutting tool may be manipulated to cut the organ along the inner edge of the aperture of the organ conformable panel, wherein the inner edge of the organ conformable panel is detected by the ultrasound probe during the procedure.

In some implementations, portions of the organ conformable panel may directly overlie those portions of an organ designated for removal. In such implementations, the cutting tool may be manipulated so as to cut through organ conformable panel and into the underlying portions of the organ designated for removal. In some implementations, the passive ultrasound detectable markers may form a line or boundary along which the cutting tool may be manipulated during the removal of the targeted portions of the organ. In some implementations, the passive ultrasound detectable marker may have a size and shape directly correspond to the size and shape of the portion of the organ designated for removal, wherein the cutting tool is manipulated to remove the marker as well as the directly underlying portion of the organ designated for removal.

In particular example implementations, the example ultrasound sensing systems further employ an optical sensor to optically capture images of the organ and the organ conformable panel. For example, a camera or endoscopic probe may be positioned within a patient such that while the organ conformable panel is being sensed by the organ surface ultrasound probe, the optical sensor is also capturing optical images of the organ conformable panel. The optical sensor or camera may be positioned within the surgical arena such that patient's organ is not between the second sensor and the organ conformable panel. Stated another way the endoscopic camera has a direct view of the organ conformable panel without the organ being between the endoscopic camera and organ conformable panel. An image processing system can distinguish the target from the surrounding material. In some implementations, the optical sensor may comprise a stereoscopic camera to provide three-dimensional coordinates and positioning of the organ conformable panel relative to the optical sensor or camera.

In some implementations during a procedure, the optical data or optical images captured by the optical sensor and the ultrasound data or ultrasound images resulting from the organ surface ultrasound probe are simultaneously or concurrently presented by a controller for viewing by a medical practitioner and/or for use by a robotic system controlling a cutting tool or other surgical tool. In some implementations, the ultrasound data and the optical data are superimposed and registered relative to one another for use by controller to carry out automatic a robotic control of a surgical tool, such as a cutting tool. In some implementations, the optical images and the ultrasound images are superimposed and registered for viewing by a medical practitioner may be controlling a cutting tool. Registration means that the portion of an ultrasound image or data corresponding to a real-world physical location on the organ will be aligned with or directly overlapping the portion of the optical image or data corresponding to the same real-world physical location on the organ.

In some implementations, optical data or optical images captured by the optical sensor includes the optically capture location of the passive ultrasound detectable marker or locations of multiple passive ultrasound detectable markers supported by the organ conformable panel. The optically captured locations of the individual markers may be registered with the location of the individual markers included in the ultrasound data or depicted in the ultrasound images. Because the particular locations of the passive ultrasound detectable markers are sensed both optically and by ultrasound, the locational accuracy of such markers may be enhanced for more precise and accurate mapping of the organ surfaced during movement of the organ and/or control over manipulation of a surgical tool interacting with particular portions of the organ, such as a tumor designated for removal.

In those implementations where the passive ultrasound detectable markers supported by the organ conformable panel are not optically visible when the organ conformable panel is positioned on the organ, the organ conformable panel may be additionally provided with an optical marker having a number and locations corresponding to the passive ultrasound detectable marker or multiple optical markers corresponding to the multiple passive ultrasound detectable markers. Such optical markers may be provided on a surface of the organ conformable panel opposite to the face of the organ conformable panel facing the organ, wherein such optical markers are have locations corresponding to the locations of the passive ultrasound detectable markers, permitting the optical markers to be optically captured by the optical sensor and permitting the location of the otherwise non-viewable passive ultrasound detectable marker or markers to be determined and tracked based upon signals from the optical sensor.

In some implementations, portions of the organ conformable panel supporting the passive ultrasound detectable marker or markers may be formed from a transparent or translucent material, permitting the passive ultrasound detectable marker or markers to be optically sensed by the optical sensor despite such passive ultrasound detectable markers being below the outer surface or face of the organ conformable panel. In some implementations, the passive ultrasound detectable marker or markers may be formed from a transparent or translucent material, whereas the remaining portions of the organ conformable panel are opaque, permitting the location of the passive ultrasound detectable marker markers to be optically determined from the signals output by the optical sensor. In such implementations, such optical markers may be omitted.

In some implementations, the organ conformable panel may be formed from a continuous panel of a biocompatible polymer or other biocompatible material having an acoustic impedance different than that of the organ upon which the organ conformable panel is to be mounted or supported. In some implementations, the organ for panel may be formed from a material such as Polyester, Polypropylene/poliglecaprone, polytetrafluorethylene condensate, polyester/collagen, polypropylene/polydioxanone/oxidized regenerated cellulose and the expanded polytetrafluorethylene. In some implementations, the organ conformable panel may be formed from a fabric or mesh. On Ultrasound, the polypropylene and the expanded polytetrafluorethylene meshes may be especially hyperechoic. A variant of the polypropylene monofilament mesh are the Prolite and Prolene Light varieties, which offer increased flexibility than their counterparts. Other metal-based options include Nitinol, a biocompatible, hyperechogenic metallic alloy; and magnesium alloy, which is less flexible. These metal meshes offer the advantages of being clearly identifiable on a wider variety of medical imaging, other than exclusively Ultrasound. In some implementations, the organ can four panel may be formed from a continuous transparent material that allows an optical sensor that allows an optical sensor to see through the material. Example of such transparent materials include, but are not limited to, Ethicon Prolene Soft Polyproylene Mesh, which is constructed of transparent knitted monofilaments of polypropylene.

In some implementations, the organ conformable panel may be formed from a material or materials that do not unravel but maintain their shape when being cut or severed. In some invitations, the organ control panel may be formed from a material or materials that are biodegradable such that any portions or strands remaining on the organ may biodegrade and not pose a risk to the patient if not removed from a surgical site. Examples of such materials include, but are not limited to, Ethicon Vicryl Mesh (Ethicon Inc., Somerville, N.J.), Phasix Mesh (C. R. Bard, Inc./Davol Inc., Warwick, R.I.), Tigr Matrix (Novus Scientific, Uppsala, Sweden), and Gore Bio-A (W.L. Gore and Associates, Inc., Flagstaff, Ariz.). All of these meshes are composed of synthetic resorbable monofilament polymers, either in a single or double layer that is gradually degraded over time. The implementations differ in their in their materials, patterns, caliber of porosity and rates of degradation—however, they are all echogenic. The passive ultrasound detectable markers may be formed from similar materials, but where such materials have a different acoustic impedance relative to the material or materials of the organ conformable panel.

For purposes of this application, the term “processing unit” shall mean a presently developed or future developed computing hardware that executes sequences of instructions contained in a non-transitory memory. Execution of the sequences of instructions causes the processing unit to perform steps such as generating control signals. The instructions may be loaded in a random-access memory (RAM) for execution by the processing unit from a read only memory (ROM), a mass storage device, or some other persistent storage. In other embodiments, hard wired circuitry may be used in place of or in combination with software instructions to implement the functions described. For example, a controller may be embodied as part of one or more application-specific integrated circuits (ASICs). Unless otherwise specifically noted, the controller is not limited to any specific combination of hardware circuitry and software, nor to any particular source for the instructions executed by the processing unit.

For purposes of this disclosure, the term “coupled” shall mean the joining of two members directly or indirectly to one another. Such joining may be stationary in nature or movable in nature. Such joining may be achieved with the two members, or the two members and any additional intermediate members being integrally formed as a single unitary body with one another or with the two members or the two members and any additional intermediate member being attached to one another. Such joining may be permanent in nature or alternatively may be removable or releasable in nature. The term “operably coupled” shall mean that two members are directly or indirectly joined such that motion may be transmitted from one member to the other member directly or via intermediate members. The term “fluidly coupled” shall mean that two or more fluid transmitting volumes are connected directly to one another or are connected to one another by intermediate volumes or spaces such that fluid may flow from one volume into the other volume.

For purposes of this disclosure, the phrase “configured to” denotes an actual state of configuration that fundamentally ties the stated function/use to the physical characteristics of the feature proceeding the phrase “configured to”.

For purposes of this disclosure, the term “releasably” or “removably” with respect to an attachment or coupling of two structures means that the two structures may be repeatedly connected and disconnected to and from one another without material damage to either of the two structures or their functioning.

For purposes of this disclosure, unless explicitly recited to the contrary, the determination of something “based on” or “based upon” certain information or factors means that the determination is made as a result of or using at least such information or factors; it does not necessarily mean that the determination is made solely using such information or factors. For purposes of this disclosure, unless explicitly recited to the contrary, an action or response “based on” or “based upon” certain information or factors means that the action is in response to or as a result of such information or factors; it does not necessarily mean that the action results solely in response to such information or factors.

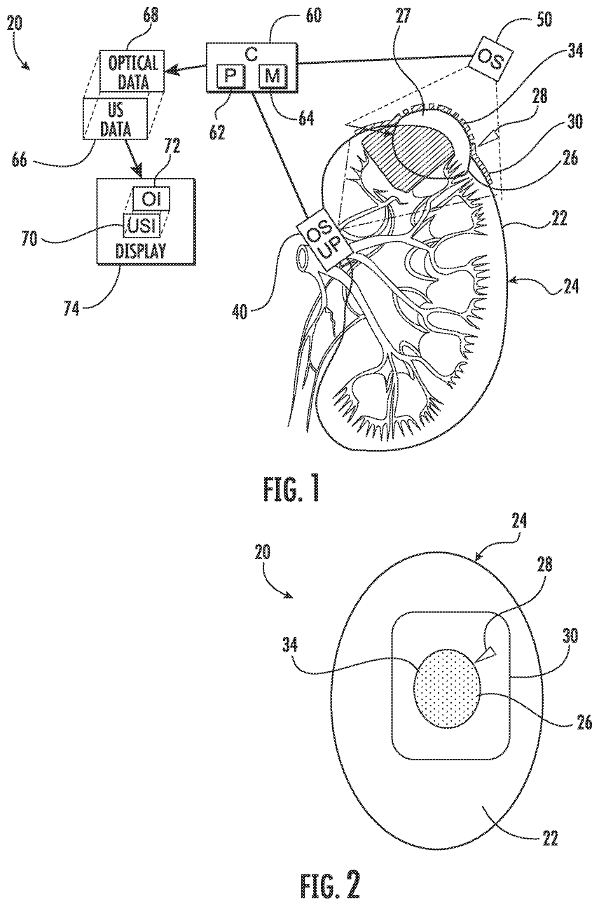

FIG. 1 is a diagram schematically illustrating portions of an example ultrasound sensing system 20 for sensing an exterior surface 22 of an organ 24 during a surgical procedure. System 20 may facilitate more precise and accurate positioning of a tool within the patient relative to the target tissue, compensating for movement of the tool and/or organ, such as movement caused by interaction of the tool with the organ or movement resulting from involuntary respiration. Although FIG. 1 illustrates an example organ 24 in the form of a kidney having an example tumor 26, at least portions of which are designated for removal by the surgical procedure, in other implementations, the organ interacted upon by system 20 may comprise other types of organs or other surgical procedures. Ultrasound sensing system 20 comprises organ conformable panel 30, passive ultrasound detectable marker 34, organ surface ultrasound probe 40, optical sensor 50, and controller 60.

Organ conformable panel 30 comprises an expanse of one or more materials configured to be positioned on a portion of exterior surface 22 of internal organ 24, while internal organ 24 resides within a patient's body. In particular implementations, panel 30 is configured to be rolled or otherwise folded for insertion into the patient's body through a cannula or other tube, wherein panel 30 is unrolled or unfolded and deployed on surface 22 by surgical tools. Organ conformable panel 30 may include an underlying adhesive layer for retaining panel 30 against and in contact with exterior surface 22. In some implementations, organ conformable panel 30 may be in the form of a gel which wets to surface 22 and retains panel 30 in contact with surface 22 of organ 24.

Organ conformable panel 30 has a material composition that is sufficiently flexible, bendable, stretchable or pliable so as to wrap over portions of exterior surface 22, conforming to the existing shape of surface 22, including any bulging portions that may be that of a tumor forming on organ 24. Panel 30 has material composition that is capable of bending, stretching, deforming or otherwise moving with the movement of surface 22 as a result of interaction of tools with surface 22 or involuntary movement of organ 24. Organ conformable panel 30 has a material composition that has an acoustic impedance (echogenicity) different than that of surface 22 or the other tissue of organ 24. Examples of the various materials from which panel 30 may be formed are set forth above.

In the example illustrated, panel 30 is sized so as to cover and extend outwardly beyond tumor 26 onto surrounding healthy tissue of organ 24. In other implementations, panel 30 may be sized and shaped such a correspond to the size and shape of the bulging or protruding portion of tumor 26. In yet other implementations, panel 30 may be sized so as to be smaller than at least one dimension of the bulging portion of tumor 26.

Passive ultrasound detectable marker 34 (schematically represented in FIG. 1 by a dashed line) is supported by panel 30. Passive ultrasound detectable marker 34 has a material composition (formed from one or more materials) that has an acoustic impedance different than the acoustic impedance of panel 30 and different from the acoustic impedance of organ 24. As a result, those ultrasound echoes reflected from marker 34 will be different and distinguishable from those ultrasound echoes reflected from panel 30. Marker 34 may be formed from a variety of materials, including the example materials set forth above for forming panel 30, but wherein the particular materials forming panel 30 and marker 34 are different so as to have different acoustic impedances.

In the example illustrated, marker 34 has a size and shape corresponding to the size and shape of the protruding portion or bulging portion of tumor 26 of tumor 26 along the exterior surface 22 of organ 24. In the example illustrated, panel 30 is positioned on organ 24 such that the outer perimeter edge of marker 34 corresponds to the outer perimeter edge of those portions of tumor 26 protruding from the remainder of organ 24. Because marker 34 may be distinguished from the remainder of panel 30 by an ultrasound probe due to the different acoustic impedances of panel 30 and marker 34, the edges of marker 34 may be used to assist in the manipulation of a surgical tool, such as cutting tool 28, when removing at least portions of tumor 26. For example, cutting tool 28 may cut through panel 30 and into surface 22 of organ 24 along a junction of marker 34 and panel 30.

Organ surface ultrasound probe 40 comprises a sensing head that is positionable and securable upon exterior surface 22 and that comprises an ultrasound transducer (sometimes referred to as an ultrasonic sensor) configured to direct ultrasound waves (ultrasonic energy or mechanical waves) towards panel 30 and to sensor to detect ultrasound echoes that have reflected from panel 30 and marker 34. In the example illustrated, organ surface ultrasound probe is located on an opposite side of panel 30 and marker 34 as optical sensor 50. In some implementations, the transducer may comprise an ultrasound transceiver, a device that may convert electrical signals into ultrasound or mechanical waves and that may receive and convert reflected or scattered ultrasound or mechanical waves into electrical signals which are then detected, conditioned, post-processed for display. Such signals, at some point, are transmitted to controller 60 in a wired or wireless fashion.

In some implementations, the transducer comprises a piezoelectric (PZT) transducer that converts alternating electrical current (AC) into ultrasound using piezoelectric crystals, wherein the AC voltage oscillates such crystals to produce the ultrasonic sound or mechanical waves. In some implementations, the transducer may comprise a capacitive transducer which generates ultrasound or mechanical waves using electrostatic fields between a conductive diaphragm and a backing plate. In some implementations, the transducer may comprise a plurality of individual transducer elements or an array of transducer elements. In some implementations, the transducer may comprise a conformable (conformable) transducer, a transducer having multiple transducer element supported by a flexible substrate. In some implementations, the transducer is acoustically coupled to surface 22 of organ 24 by an acoustic coupler having and acoustic coupling agent or medium with an acoustic impedance similar to that of surface 22 and organ 24 to enhance the transmission of ultrasound waves between the transducer and an interior of organ 24. Example organ surface ultrasound probes are described and depicted in U.S. Application No. 63/332,652 entitled ULTRASOUND SENSING SYSTEM filed on Apr. 19, 2022 and PCT Application No. PCT/US2022/040128 entitled ULTRASOUND SENSING SYSTEM filed on Aug. 11, 2022 both of which are incorporated by reference in their entirety.

Optical sensor 50 comprise a device configured to optically sense optically visible wavelengths of light reflected from organ conformable panel 30, marker 34 and surrounding portions of organ 24. Optical sensor 50 is positioned within the anatomy of a patient, such as within an abdominal cavity, so as to have a field-of-view encompassing panel 30 and marker 34. In some implementations, optical sensor 50 comprises a camera, such as a two-dimensional camera or a stereoscopic camera. In some implementations, optical sensor 50 is part of an endoscopic probe. Optical sensor 50 is configured to optically capture the positioning and, in some implementations, the movement, of panel 30 and marker 34 of organ 24. Signals from optical sensor 50 are transmitted to controller 60 in a wired or wireless fashion. In some implementations, optical sensor 50 may be omitted, wherein system 20 operates without optical sensor 50.

Controller 60 receives signals from probe 40 and sensor 50 and utilizes such signals to monitor organ 24 during a surgical procedure. Controller 60 comprises processing unit 62 and a non-transitory computer-readable medium in the form of memory 64. Memory 64 contains computer-readable instructions for directing processing unit 62 to output control signals controlling the operation of probe 40 and sensor 50 and to store and utilize ultrasound data from probe 40 and optical data from optical sensor 50. Such data may be used to assist a medical practitioner in the control and positioning of a surgical tool, since cutting tool 28 and/or may be used by an automated robotic system which controls aspects (positioning, power, cutting rate and the like) pertaining to the use of a surgical tool such as cutting tool 28.

As schematically depicted by FIG. 1, controller 60 may superimpose and register the ultrasound data 66 received from probe 40 and the optical data 68 received from optical sensor 50. Registration means that the portion of an ultrasound image or data corresponding to a real-world physical location on the organ will be aligned with or directly overlapping the portion of the optical image or data corresponding to the same real-world physical location on the organ. As further schematically depicted in FIG. 1, controller 60, in some implementations, may register an ultrasound image 70 generated from or based upon the ultrasound data from probe 40 and an optical image 72 generated from or based upon the image data received from optical sensor 50. The registered images 70 and 72 may be transmitted in a wired or wireless fashion to a display 74 or viewing by a medical practitioner or others. Such registered data 66, 68 or images 70, 72 may be stored in memory 64 or in an alternative non-transitory computer-readable medium or memory.

FIGS. 3 and 4 illustrate an example organ conformable panel 130 supporting an example passive ultrasound detectable marker 134. Panel 130 and marker 134 may be employed in place of panel 30 and marker 34 in system 20 described above. As with such previously described components, panel 130 and marker 134 have different acoustic impedances such that they may be identified and distinguished from one another based upon the ultrasound echoes received by probe 40.

FIG. 4 is a sectional view of the panel/marker combination shown in FIG. 3 taken along line 4-4 of FIG. 3. As shown by FIG. 4, marker 134 is supported within and is surrounded by panel 130. Marker 134 extends through panel 130 from a first face 136 which is to be positioned against surface 22, either directly or by an intervening adhesive or acoustic coupling medium, to a second opposite face 138 which is to face the optical sensor 50. In some implementations, marker 134 has a size and shape corresponding to or based upon the size and shape of the protruding portion of tumor 26 of a tumor 26 of organ 24. In other implementations, marker 134 has a size and shape corresponding to a portion of the organ/tumor designated for removal. Because marker 134 extends along face 138, marker 134 is optically visible to optical sensor 50 such that the positioning of marker 134 may be identified and monitored by both probe 40 and sensor 50. Although panel 130 and marker 134 our illustrated with generally rectangle and oval shapes, respectively, in other implementations, panel 130 and/or marker 134 may have other shapes. In some implementations, panel 130 may include multiple spaced or distinct markers 134.

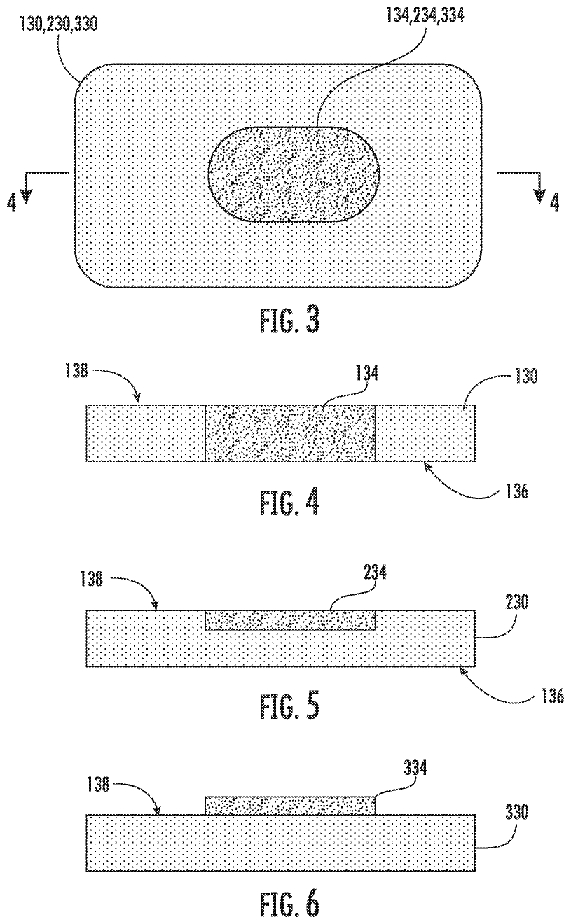

FIG. 5 is a sectional view of an example organ conformable panel 230 and passive ultrasound detectable marker 234 taken along line 4-4 of FIG. 3. Panel 230 and marker 234 are similar to panel 130 and marker 134 except that marker 234 is recessed within face 138 of panel 230 and does not extend completely through panel 230 to face 136. As with marker 134, marker 234 is visible to optical sensor 50 and is detectable by probe 40 due in part to its acoustic impedance which differs from that of panel 230.

FIG. 6 is a sectional view of an example organ conformable panel 330 and passive ultrasound detectable marker 334 taken along line 4-4 of FIG. 3. Panel 330 and marker 334 are similar to panel 130 and marker 134 except that marker 234 resides on top of face 138 of panel 330. In some implementations, marker 324 may be adhered, fused or otherwise joined to face 138. In some implementations, marker 334 may be coated upon face 138, such as by painting, printing, spraying or other application techniques. As with marker 134, marker 334 is visible to optical sensor 50 and is detectable by probe 40 due in part to its acoustic impedance which differs from that of panel 330.

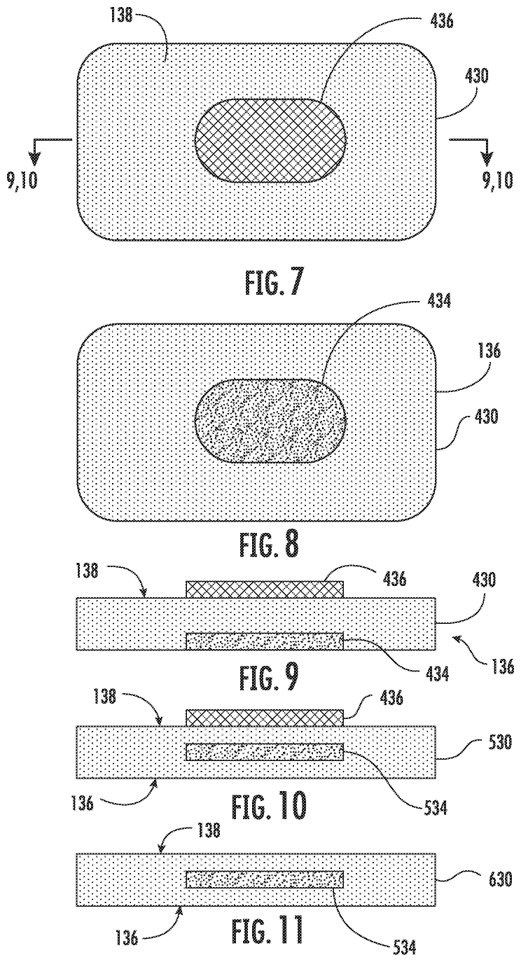

FIGS. 7, 8 and 9 illustrate an example organ conformable panel 430 and passive ultrasound detectable marker 434 for use as part of system 20. As shown by FIGS. 7 and 9, panel 430 additionally supports an optical marker 436. As shown by FIG. 9, marker 434 is recessed or embedded within the lower face 136 of panel 430, whereas optical marker 436 is positioned on top of face 138 of panel 430. In other implementations, optical marker 436 may alternatively be recessed or partially embedded into face 138 of panel 430. Because passive ultrasound detectable marker 434 extends along the lower face 136 and does not extend completely through panel 430, in those implementations where panel 430 is opaque, marker 434 may not be visible to optical sensor 50 when positioned on organ 24 (shown in FIG. 1). However, because optical marker 436 is at least partially exposed along face 138, optical marker 436 is visible to optical sensor 50. Because optical marker 436 has a location which indicates the underlying location of marker 434, optical sensor 50 may identify the current location of marker 434 by determining the location of marker 436.

Optical marker 436 comprises any marking which is exposed or optically viewable from face 138 by optical sensor 50 (shown in FIG. 1). Optical marker 436 indicates or provides optical information from which the location of marker 434 may be determined. In the example illustrated, optical marker 436 has a shape corresponding to marker 434 and is aligned with marker 434. In some implementations, optical marker 436 may alternatively comprise a line which is aligned with and corresponds to the outer peripheral shape of marker 434. In some implementations, optical marker before 36 may comprise an arrow, “X” pointing to or indicating a predetermined point of the underlying marker 434 or a predetermined location having a predefined spatial relationship to marker 434.

Although illustrated as relatively thick for purposes of illustration, marker 436 may comprise a thin film or coating applied to face 138. The coating may be applied before after panel 430 is secured to surface 22 of organ 24. In some implementations, marker 436 may be applied by spraying, printing, painting or drawing on face 138. In some implementations, marker 436 may be fused, welded, adhesively bonded or otherwise joined to panel 430. In some implementations, marker 436 and/or marker 434 may be co-molded with panel 430. In some implementations, marker 436 may be partially or completely embedded into or recessed into face 138.

FIG. 10 is a sectional view of an example organ conformable panel 530, passive ultrasound detectable marker 534 and optical marker 436. In one implementation, the top view of the apparatus shown in FIG. 10 would be similar to the view shown in FIG. 7. Panel 530 and marker 534 are similar to panel 430 and marker 434, respectively, described above except that passive ultrasound detectable marker 534 is completely encapsulated or embedded within the material composition forming panel 530. In one implementation, no portion of marker 534 is exposed on face 136. Although the material composition panel 530 is opaque, hindering the direct optical detection of marker 534 by optical sensor 50 (shown in FIG. 1), optical marker 436 facilitates the optical detection of the current position of marker 534.

FIG. 11 is a sectional view of an example organ conformable panel 630 and passive ultrasound detectable marker 534. The apparatus shown in FIG. 11 is similar to the apparatus shown and described with respect to FIG. 10 except for the omission of optical marker 436. As with each of the implementation shown in FIGS. 3-10, the organ conformable panel has a first acoustic impedance while the passive ultrasound detectable marker has a second different impedance, facilitating their distinction based upon the ultrasound echoes received by probe 40. In the example shown in FIG. 11, organ conformable panel 630 has a transparent material composition such that the location of marker 534 may be optically determined based upon signals from optical sensor 50. Examples of such transparent materials for panel 630 include, but are not limited to, Ethicon Prolene Soft Polyproylene Mesh, which is constructed of transparent knitted monofilaments of polypropylene.

In some implementations, panel 630 may be formed from a transparent gel or hydrogel which encapsulates marker 534 and which has a wettable lower face 136 to facilitate bonding to surface 22 of organ 24. Each of the above-described implementations may likewise utilize an organ conformable panel that comprises a gel or hydrogel for securement to surface 22 of organ 24. As described above, each of the above-described organ conformable panels in each of the implementations may alternatively comprise an adhesive layer across face 136 for securing the organ conformable panel to surface 22 of organ 24. In some implementations, the adhesive forming the adhesive layer is configured to be acted upon by solvent for when the organ conformable panel is to be released from surface 22 of organ 24.

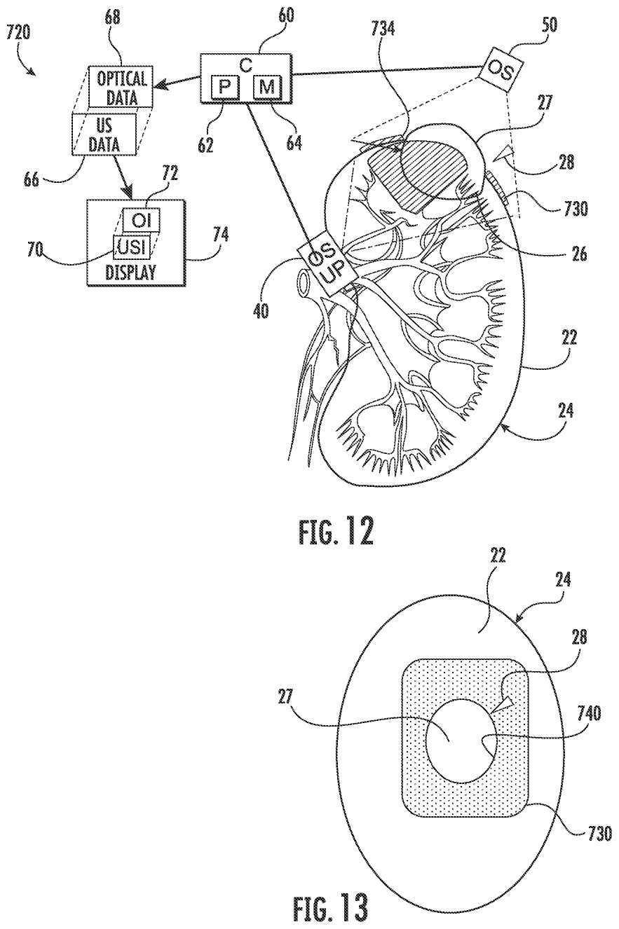

FIG. 12 is a diagram illustrating portions of an example ultrasound sensing system 720. FIG. 12 illustrates an example of how an organ conformable panel secured to the surface of an organ may be used as a cutting template for removing portions of the organ. System 720 is similar to system 20 described above except that system 720 comprises organ conformable panel 730 in place of panel 30 and omits passive ultrasound detectable marker 34. Those remaining components of system 720 which correspond to components of system 20 are numbered similarly and/or are shown and described above with respect to system 20.

Organ conformable panel 730 comprises a panel formed from a material composition having an acoustic impedance different than that of surface 22 or the tissue of organ 24. Organ conformable panel 730 is similar to organ conformable panel 30 described above except that organ conformable panel comprises an aperture 734 which extends completely through panel 730 and which is bounded on all sides by an internal edge 740 of panel 730. As shown by FIGS. 12 and 13, panel 730 is sized and shaped so as to extend beyond those portions of organ 24 designated for removal. Aperture 734 is sized, shaped and located so as to extend over those portions of organ 24 designated for removal, such as portions of tumor 26. In some implementations where those portions of organ 24 designated for removal are bulging or protruding, such portions may protrude within or even through aperture 734. In the example illustrated, aperture 734 is sized, shaped and located such that its internal edge 740 coincides with or slightly outside of an intended cutting path or cutting line for a cutting tool 28. In some implementations, the internal edge 740 extends about and outlines an entirety of the portion of the organ 24 designated for removal.

Organ conformable panel 730 serves as a cutting template for use during a surgical procedure removing portions of organ 24, such as tumor 26. In those implementations in which aperture 740 receives an entirety of exposed portion of the organ 24 designated for removal and wherein the shape and size of aperture 740 corresponds to the shape and size of the organ portion designated for removal, organ conformable panel 730 serves four concurrent functions. First, edges 740 may be detected by ultrasound probe 40 to delineate those portions designated for removal from those portions not designated for removal. Second, edges 740 may be optically detected by optical sensor 50 to further delineate those portions designated for removal (within aperture 734) from those portions not designated for removal. Aperture 734 permits optical sensor 50 to provide optical imagery of the exposed portion of the tumor during its removal. Third, organ conformable panel 730 provides edge 740 for guiding the movement of cutting tool 28 therewithin and proximate to edge 740 for cutting and removing portions of organ 24. Fourth, those portions of panel 730 outside of edges 740 overlie tissue of organ 24 which is not to be removed, such as healthy tissue. Such portions of panel 730 may inhibit or reduce the likelihood of cutting tool 28 cutting into such healthy tissue. To do so might otherwise require cutting tool 28 to cut through panel 730. In some implementations, panel 730 is sufficiently thick and/or formed from a material which prevents cutting tool 28 from completely cutting through the thickness of panel 730 about opening 734. As a result, panel 730 may serve as a safeguard that protects against accidental cutting and removal of healthy tissue.

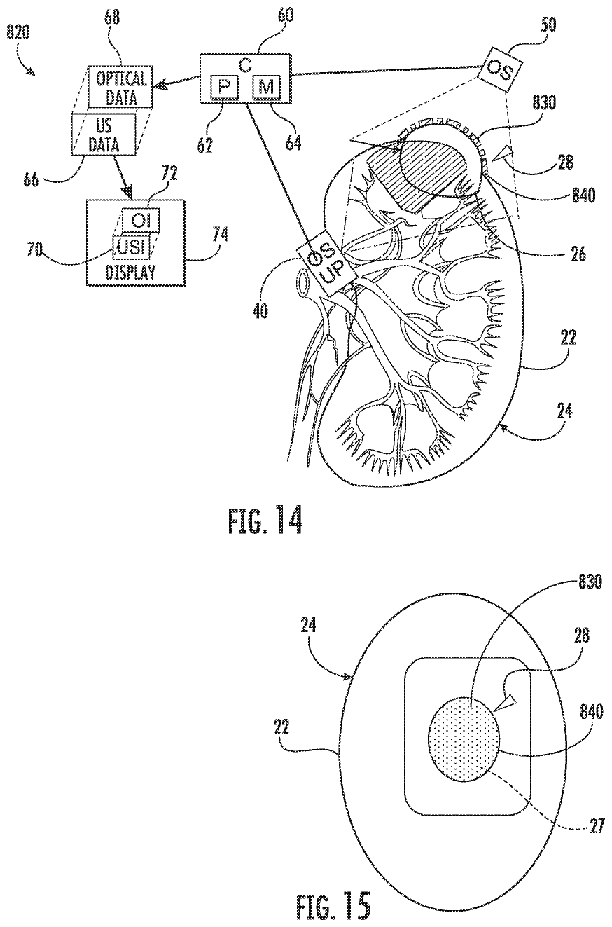

FIGS. 14 and 15 are diagrams illustrating portions of an example ultrasound sensing system 820. FIGS. 14 and 15 illustrate an example of how an organ conformable panel secured to the surface of an organ may be used as a cutting template for removing portions of the organ. System 820 is similar to system 720 described above except that system 820 comprises organ conformable panel 830 in place of panel 730. Those remaining components of system 820 which correspond to components of system 720 are numbered similarly and/or are shown and described above with respect to systems 20 and/or 720.

Organ conformable panel 830 comprises a panel formed from a material composition having and acoustic impedance different than that of surface 22 or the tissue of organ 24. Organ conformable panel 830 is similar to organ conformable panel 730 except that organ conformable panel 830 is sized and shaped so as to closely correspond to those portions of organ 24 designated for removal. Panel 830 has an outer edge 840 sized, shaped and located so as coincide or slightly lie to the inside or outside of the exposed perimeter portions of organ 24 designated for removal, such as portions of tumor 26. In some implementations where those portions of organ 24 designated for removal are bulging or protruding, edge 840 may extend along the base of the bulging or protruding portion of tumor 26.

Organ conformable panel 830 serves as a cutting template for use during a surgical procedure removing portions of organ 24, such as tumor 26. In those implementations in which the shape and size of panel 830 correspond to the shape and size of the organ portion designated for removal and in which panel 830 is positioned on organ 24 in alignment with those portions of organ 24 designated for removal, organ conformable panel 730 serves three concurrent functions. First, edges 840 may be detected by ultrasound probe 40 to delineate those portions designated for removal from those portions not designated for removal. Second, edges 840 may be optically detected by optical sensor 50 to further delineate those portions designated for removal (those portions underlying panel 830 within edges 840) from those portions not designated for removal (those portions outside of edges 840). Panel 830 permits optical sensor 50 to provide optical imagery of the healthy tissue not designated for removal and extending adjacent to tumor during its removal. Third, organ conformable panel 830 provides edge 840 for guiding the movement of cutting tool 28 proximate to edge 840 for cutting and removing portions of organ 24.

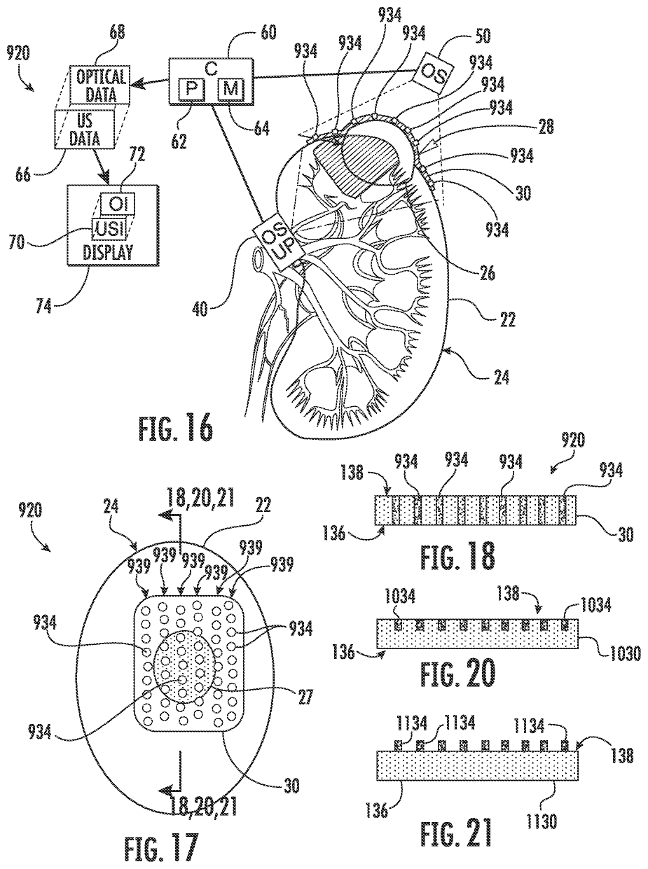

FIGS. 16-18 illustrate portions of an example ultrasound sensing system 920. FIG. 16-18 illustrate an example of how an organ conformable panel may support multiple passive ultrasound detectable markers to facilitate mapping of an organ surface. System 920 is similar to system 20 described above except that organ conformable panel 30 supports multiple passive ultrasound detectable markers 934. Those remaining components of system 920 which correspond to components of system 20 are numbered similarly and/or are shown and described above with respect to system 20.

Similar to the individual passive ultrasound detectable marker 34 described above, each of the passive ultrasound detectable markers 934 has an acoustic impedance different than that of panel 30 and different than that of surface 22 and the tissue forming organ 24. As shown by FIG. 16, the example passive ultrasound detectable markers 934 extend across a sufficient area of panel 30 such that markers 934 may be located over those portions of organ 24 designated for removal (tumor 26) and additionally over those portions of organ 24 not designated for removal, beyond tumor 26. Because each of the individual markers 934 are spaced from one another and have an acoustic impedance different than that of panel 30, the individual markers 934 may be individually identified from the ultrasound data acquired by probe 40 and may be distinguished from one another. Because each individual marker 934 has a predefined or prescribed location on panel 30, the location of each of markers 934 may likewise correspond to the underlying locations of organ. As a result, movement of the individual markers 934 may be used to track movement of the corresponding underlying locations of organ 24 in at least one dimension across surface 22.

As shown by FIG. 17, in one example implementation, markers 934 are arranged in a two-dimensional array across the expanse of panel 30. As a result, movement of the individual markers 94 may be used to track movement of the corresponding underlying locations of organ 24 in two dimensions across surface 22. In the example illustrated, markers 934 are arranged in a plurality of columns 939 of spaced markers 934. In the example illustrated, the columns 939 are vertically staggered relative to one another such that adjacent markers 934 of different columns 939 are offset or staggered relative to one another. This staggering of markers 934 may provide enhanced mapping of surface 22. In other implementations, markers 934 may be arranged in other patterns across the expanse of panel 30.

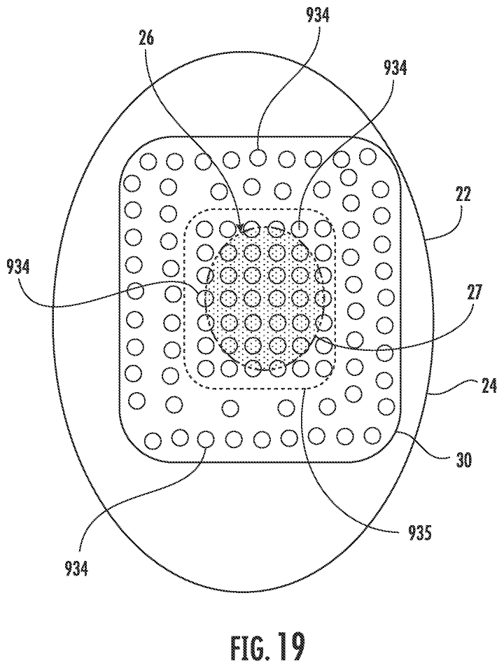

Although markers 934 are illustrated as having a generally uniform density across panel 30, in some implementations, markers 934 may have different regions of different densities of markers 934. For example, as shown by FIG. 19, in some implementations, more central portions of panel 30 may be provided with a greater density of markers 934. Panel 30 supports a region 935 (outlined by dash-dot-dash lines) which contains a greater density of markers 934 compared to those other regions of panel 30 outside of region 935. In the example shown in FIG. 19, panel 30 is positioned on organ 24 such that region 935 directly overlies tumor 26 of organ 24 and extends beyond the protruding base or boundary of tumor 26. The greater density of markers 934 over tumor 26 may provide enhanced mapping resolution for tumor 26 and for the borders of tumor 26. The enhanced mapping resolution along the borders of tumor 26 may provide enhanced, more precise control over cutting tool 28 as it is moved along the cutting path along the border of tumor 26.

As shown by FIG. 18, each of markers 934 is similar to marker 134 described above in that each of markers 934 extends completely through the thickness of panel 30, from face 136 (to be positioned against surface 22, directly or with an adhesive) to face 138. As a result, each of markers 934 is also optically visible to optical sensor 50, permitting the location of each of markers 924 to be optically determined in addition to the determination of such locations based upon ultrasound data acquired by probe 40.

In the example illustrated, some of markers 934 are additionally provided with acoustic impedances and/or optically detectable appearances (color, darkness, shape, pattern or the like) that are different with respect to the other markers 934, facilitating individual identification of individual markers 934 based on ultrasound data and/or based on optical data sensing such differing characteristics amongst the different markers 934. In the example illustrated, the markers 934 of each of columns 939 alternate between two different material compositions having different acoustic impedances. In the example illustrated, the odd markers 934 (going left to right in FIG. 18) have a first material composition having a first acoustic impedance different than that of panel 30, organ 24 and the even markers 934 in the same column. Even markers 934 and the same column have a second acoustic impedance different than that of panel 30, organ 24 and the odd markers 934 in the same column. In other implementations, different passive ultrasound detectable markers 934 may be provided with different optically detectable appearances and/or different acoustic impedances in other fashions, such as with different patterns or layouts of different markers 934. In some implementations, each of markers 934 may have the same optical appearance and acoustic impedance.

FIG. 20 is a sectional view of an example organ conformable panel 1030 and a plurality of passive ultrasound detectable markers 1034 taken along line 20-20 of FIG. 17. Panel 1030 and markers 1034 are similar to panel 30 and marker 934 except that markers 1034 are recessed within face 138 of panel 1030 and do not extend completely through panel 1030 to face 136. As with markers 934, each of markers 1034 are visible to optical sensor 50 and are detectable by probe 40 due in part to their acoustic impedance which differs from that of panel 1030.

FIG. 21 is a sectional view of an example organ conformable panel 1130 and passive ultrasound detectable markers 1134 taken along line 21-21 of FIG. 17. Panel 1130 and markers 1134 are similar to panel 1030 and markers 1034 except that markers 1134 reside on top of face 138 of panel 1130. In some implementations, markers 1134 may be adhered, fused or otherwise joined to face 138. In some implementations, marker 334 may be coated upon face 138, such as by painting, spraying, printing or other application techniques. As with markers 1034, markers 1134 are visible to optical sensor 50 and are individually detectable by probe 40 due in part to their acoustic impedances which differ from that of panel 330. As with markers 1034, some of markers 1134 have different acoustic impedances than other of markers 1134, further facilitating the distinguishing between different individual markers 1134 based upon ultrasound data.

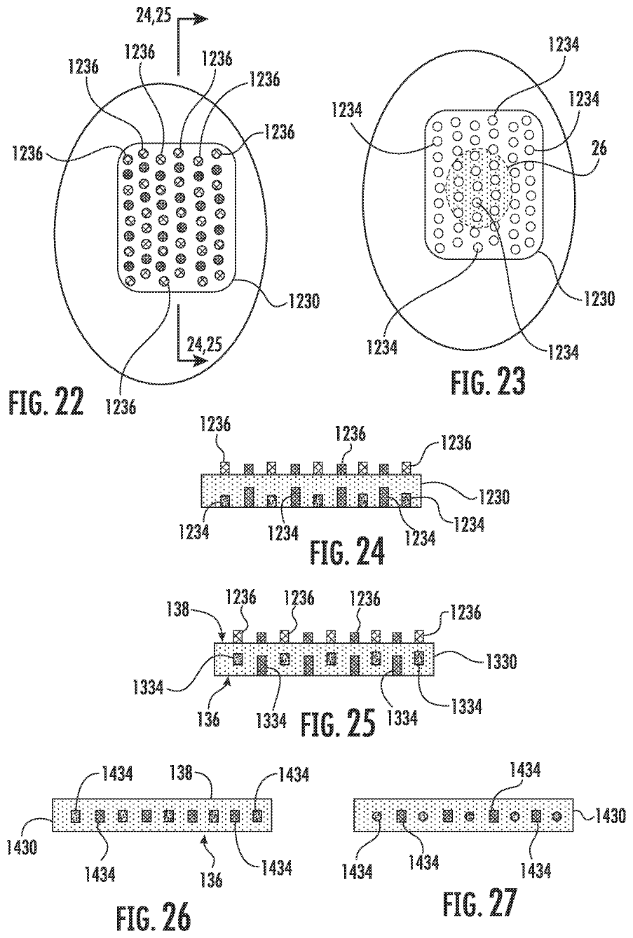

FIGS. 22, 23 and 24 illustrate an example organ conformable panel 1230 and passive ultrasound detectable markers 1234 for use as part of system 20. As shown by FIGS. 22 and 24, panel 1230 additionally supports optical markers 1236. As shown by FIG. 24, markers 1234 are recessed or embedded within the lower face 136 of panel 1230, whereas optical markers 1236 are positioned on top of face 138 of panel 1230. In other implementations, optical markers 1236 may alternatively be recessed or partially embedded into face 138 of panel 1230. Because passive ultrasound detectable markers 1234 extend along the lower face 136 and do not extend completely through panel 1230, in those implementations where panel 1230 is opaque, markers 1234 may not be visible to optical sensor 50 when positioned on organ 24 (shown in FIG. 16). However, because optical markers 1236 are at least partially exposed along face 138, optical markers 1236 are visible to optical sensor 50. Because optical markers 1236 each have a location which indicates the underlying location of shape corresponding marker 434, optical sensor 50 may identify the current location of each of markers 1234 by determining the location of each of markers 1236.

Optical markers 1236 comprises any marking which is exposed or optically viewable from face 138 by optical sensor 50 (shown in FIG. 1). Each optical marker 1236 indicates or provides optical information from which the location of irrespective corresponding marker 1234 may be determined. In the example illustrated, each optical marker 1236 has a shape corresponding to associated underlying marker 1234 and is aligned with the particular marker 1234. In some implementations, each optical marker 1236 may alternatively comprise a line which is aligned with and corresponds to the outer peripheral shape of marker 1234. In some implementations, each optical marker 1236 may comprise an arrow, “X” pointing to or indicating a predetermined point of the underlying marker 1234 or a predetermined location having a predefined spatial relationship to the assigned marker 1234. In some implementations, different optical markers 1236 may be provided with different characteristics, color, shape, tone or the like (as schematically illustrated by the different types of hatching), further facilitating the optical images captured by optical sensor 50 to optically distinguish the different optical markers 1236 from one another.

Although illustrated as relatively thick for purposes of illustration, markers 1236 may comprise a thin film or coating applied to face 138. The coating may be applied before or after panel 1230 is secured to surface 22 of organ 24. In some implementations, markers 1236 may be applied by spraying, painting, printing or drawing on face 138. In some implementations, markers 1236 may be fused, welded, adhesively bonded or otherwise joined to panel 1230. In some implementations, markers 1236 and/or markers 1234 may be co-molded with panel 1230. In some implementations, markers 1236 may be partially or completely embedded into or recessed into face 138.

FIG. 25 is a sectional view of an example organ conformable panel 1330, passive ultrasound detectable markers 1334 and optical markers 2336. In one implementation, the top view of the apparatus shown in FIG. 25 would be similar to the view shown in FIG. 22. Panel 1330 and markers 1334 are similar to panel 1230 and markers 1234 described above except that passive ultrasound detectable markers 1334 either completely encapsulated or embedded within the material composition forming panel 1330 or extend from lower face 138 into panel 1330. In one implementation, no portion of markers 1334 is exposed on face 138. Although the material composition panel 1330 is opaque, inhibiting the direct optical detection of markers 1334 by optical sensor 50 (shown in FIG. 1), optical markers 1236 facilitate the optical detection of the current position of markers 1334.

FIG. 26 is a sectional view of an example organ conformable panel 1430 and passive ultrasound detectable markers 1434. FIG. 27 is a top view illustrating the column of passive ultrasound detectable markers 1434 shown in FIG. 26. The apparatus shown in FIGS. 26 and 27 is similar to the apparatus shown and described with respect to FIG. 25 except that each of the markers 1436 is embedded artfully encapsulated within the material composition of panel 1430 and that optical markers 1236 are omitted. As with each of the implementation shown in FIGS. 18-27, the organ conformable panel has a first acoustic impedance while the passive ultrasound detectable markers each have a second different impedance, facilitating their distinction based upon the ultrasound echoes received by probe 40. In the example shown in FIGS. 26 and 27, organ conformable panel 1430 has a transparent material composition such that the location of each of markers 1436 may be optically determined based upon signals from optical sensor 50. Examples of such transparent materials for panel 630 include, but are not limited to, Ethicon Prolene Soft Polyproylene Mesh, which is constructed of transparent knitted monofilaments of polypropylene.

In some implementations, panel 1430 may be formed from a transparent gel or hydrogel which encapsulates markers 1436 and which has a wettable lower face 136 to facilitate bonding to surface 22 of organ 24. Each of the above-described fermentations may likewise utilize an organ conformable panel that comprises a gel or hydrogel for securement to surface 22 of organ 24. As described above, each of the above-described organ conformable panels in each of the implementations may alternatively comprise an adhesive layer across face 136 for securing the organ conformable panel to surface 22 of organ 24. In some implementations, the adhesive forming the adhesive layer is configured to be acted upon by solvent for when the organ conformable panel is to be released from surface 22 of organ 24.

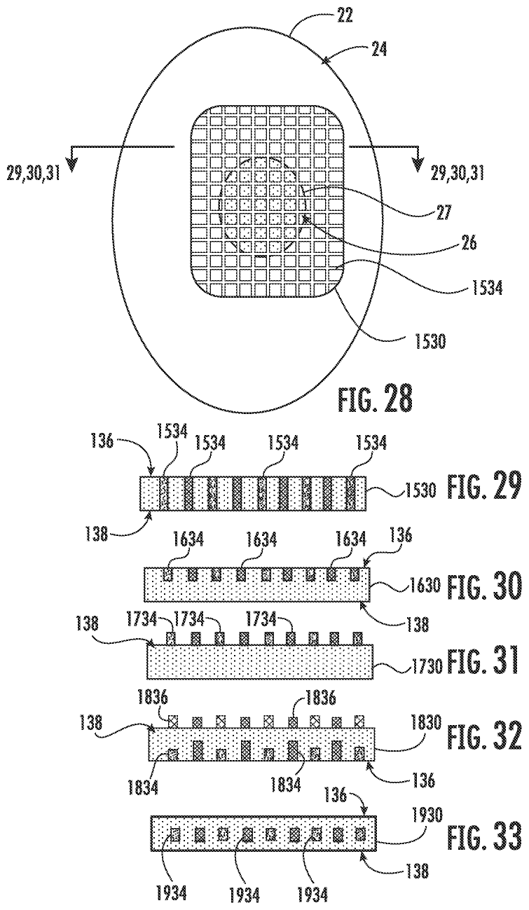

FIGS. 28 and 29 illustrate an example organ conformable panel 1530 and passive ultrasound detectable markers 1534 positioned on an external surface 22 of an organ 24, with the panel 1530 laid over an underlying tumor 26. The apparatus shown in FIGS. 28 and 29 may be employed as part of the ultrasound sensing system 920 shown and described above with respect to FIG. 16. Panel 1530 and markers 1534 are similar to panel 30 and markers 934 described above with respect to FIGS. 16-18 except that instead of distinct individual spaced points laid out in a two-dimensional array, markers 1534 comprise continuous or semi-continuous (dashed or the Ike) lines that crisscross one another to form a two-dimensional grid. Each of the lines has a material composition having an acoustic impedance different than that of the material composition of organ conformable panel 1530. As a result, each of such lines may be sensed and located based upon ultrasound data acquired by probe 40 (shown in FIG. 16). Such lines forming markers 1534 may be used to map corresponding particular locations of the underlying organ 24.

In some implementations, two-dimensional grid may be formed by a series of crisscrossing fibers, threads or the like, forming a fabric or mesh. For example, in some implementations, the horizontal gridlines may be in the form of a weft while the vertical gridlines are in the form of a warp. In some implementations, the weft and the warp may be formed from a similar material, wherein the points at which the weft and warp cross one another have a greater acoustical impedance as a result of the effective double thickness of the junction. In contrast, those portions of the panel where the weft a warp do not crisscross one another may have a lower acoustic impedance. In such implementations, the individual points formed by the crisscross of the weft and warp may appear as a 2D grid of points on the acoustic image captured by the ultrasound probe. In yet other implementations, the weft may be formed from a first material composition having a first acoustic impedance while the warp is formed from a second material composition having a second different acoustic impedance. In such implementations, the different acoustic impedances of the weft and the warp may further facilitate the detection of longitudinal and latitudinal locations along the surface of the organ conformable panel 1530.

As shown by FIG. 29, in one example implementation, each of such lines extends completely through panel 1530 from face 138 to face 136. As with markers 934, markers 1534 may be visibly identified by optical sensor 50 which captures images of face 136. Thus, the positioning of markers 1534 may be cross checked by a comparison of the optical data and the ultrasound data for enhance accuracy. In the example illustrated, different lines forming markers 1534 may be provided with different acoustic impedances and/or different visible differences (different line thicknesses, colors or the like), facilitating such different gridlines to be distinguished from one another based upon ultrasound data from probe 40 and/or optical data from optical sensor 50.

FIGS. 30-31 are sectional views illustrating variations of the passive ultrasound detectable markers 1534 shown in FIGS. 28 and 29. FIG. 30 illustrates an organ conformable panel 1630 which supports passive ultrasound detectable marker 1634. Markers 1634 are similar to markers 1534 except that markers 1634 project partially into face 136 and do not extend completely through the thickness of panel 1630.

FIG. 31 illustrates an organ conformable panel 1730 supporting passive ultrasound detectable marker 1734. Markers 1734 are similar to markers 1534 except that markers 1734 reside on the top face 138 of panel 1730. In each of the implementation shown in FIGS. 30 and 31, the organ conformable panel 1630, 1730 having acoustic impedance that is different than that of the tissue of organ 24 and that is also different than that of the markers 1634, 1734. In the example illustrated, different marker 1634, 1734 may be provided with different acoustic impedances and size or different visible characteristics to further facilitate distinguishing between such individual markers 1734 which are in the form of the grid of lines shown in FIG. 28.

FIG. 32 is a sectional view illustrating organ conformable panel 1830, passive ultrasound detectable markers 1834 and optical markers 1836. Markers 1834 are located on the lower face 136 or are recessed in the lower face 136 of panel 1830. In those implementations panel 1830 is formed from an opaque material, markers 1834 not visibly detectable from face 138 or by optical sensor 50 (shown in FIG. 16). However, optical markers 1836 are provided on face 138 at locations corresponding to or otherwise indicating the underlying locations of corresponding markers 1834. In some implementations, optical markers 1836 comprise a crisscross of gridlines similar to that shown in FIG. 28 which directly overlie, correspond to and are aligned with the underlying gridlines formed by markers 1834. As a result, optical sensor 50 may visibly detect the location of optical markers 1836 two facilitate the determination of the corresponding underlying physical locations of markers 1834.

In some implementations, optical markers 1836 may be fused, welded or otherwise adhered to face 138. In other implementations, the gridlines forming optical markers 1836 may be coated, sprayed, drawn or printed upon face 138. As discussed above, panel 1830 has a material composition having a first acoustic impedance, whereas markers 1834 have at least one second acoustic impedance different than the first acoustic impedance. In some implementations, different markers (different gridlines) 1834 have different acoustic impedances, facilitating the distinguishing of the different markers 1834 (the different gridlines shown in FIG. 28) based upon ultrasound data acquired by probe 40.

FIG. 33 is a sectional view illustrating organ conformable panel 1930 and passive ultrasound detectable markers 1934. Panel 1930 is similar to panel 1530 described above except that markers 1934 are completely embedded or encapsulated within the material composition of panel 1930. Markers 1934 are themselves similar to markers 1534 in that they form a series of crisscrossing lines forming a two-dimensional grid across the expanse of panel 1930 similar to that shown in FIG. 28. The two-dimensional grid facilitates mapping of the underlying exterior surface 22 of organ 24 using ultrasound data acquired by probe 40.

In the example illustrated, panel 1930 is formed from a transparent material composition. As a result, the particular locations of the gridlines forming markers 1934 may be visibly detected by optical sensor 50 which faces face 138. Because the different gridlines formed by markers 1934 are visible to optical sensor 50, the underlying exterior surface 22 of organ 24 may be additionally optically mapped using such gridlines. The optically determined gridlines and the ultrasound determined gridlines may be registered to register the data and/or imagery acquired from both the ultrasound and optical sensing of organ 24.

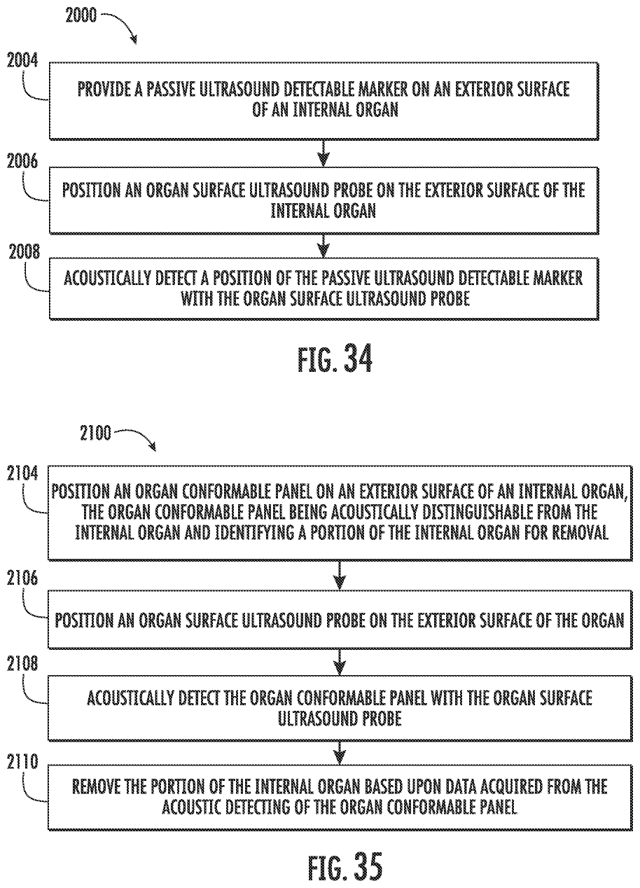

FIG. 34 is a flow diagram of an example method 2000 for tracking or monitoring movement of an organ surface during a medical procedure on in organ. Method 2000 may be carried out with any of the above-described ultrasound sensing systems, organ conformable panels and passive ultrasound detectable markers. Likewise, method 2000 may be carried out with other similar systems.

As indicated by block 2004, a passive ultrasound detectable marker is provided on an exterior surface of an internal organ. The passive ultrasound detectable marker may be provided on an organ conformable panel which is positioned on an exterior surface of the organ. The organ conformable panel may have an adhesive surface or an adhesive layer to adhesively secure the panel to the organ surface. In some implementations, the organ conformable panel may comprise a wettable surface, such as a gel or hydrogel which adheres to the organ surface.

In some implementations, the passive ultrasound detectable marker may be directly applied to the organ surface. For example, in some implementations, the passive ultrasound detectable marker may be sprayed onto the internal organ.

The passive ultrasound detectable marker may indicate underlying portions of the organ designated for treatment or removal. For example, the marker may comprise an arrow, bull's-eye or the like indicating those portions of the underlying organ that are designated for removal or indicating those portions of the underlying organ which are not to be removed, identifying particular portions of the organ where a cutting procedure should be adjusted to reduce the likelihood of accidental cutting of tissue. In some implementations, the passive ultrasound detectable marker may be in the form of a two-dimensional array of individual markers or a grid of crisscrossing lines. The passive ultrasound detectable markers may be in the form of an array or grid may facilitate mapping of the underlying organ surface using ultrasound data acquired by an ultrasound probe, such as an organ surface ultrasound probe.

As indicated by block 2006, an organ surface ultrasound probe is positioned on an exterior surface of the internal organ. The organ surface ultrasound probe is positioned such that its field-of-view encompasses the passive ultrasound detectable marker (or markers) while on the exterior surface of the internal organ. In some implementations, the probe is positioned such to capture the organ conformable panel supports the passive ultrasound detectable markers.

As indicated by block 2008, the organ surface ultrasound probe is used to acoustically detected a position of the passive ultrasound detectable markers. As noted above, the ultrasound data acquired by the organ surface ultrasound probe may be used to identify particular portions of the internal organ underlying the passive ultrasound detectable markers for removal or for protection during a removal procedure. As noted above, in some implementations, the ultrasound data acquired by the organ surface ultrasound probe may be used to map the underlying organ, wherein the map may be used to control and guide a cutting tool. The map may be used to assist a medical practitioner in controlling movement of a cutting tool or may be used by an automated robotic surgical system which moves the cutting tool, or which may position control other associated tools during the medical procedure.

FIG. 35 is a flow diagram of an example method 2100 for removing a portion of an internal organ, such as a tumor. Method 2100 may be utilized with any of the above-described ultrasound sensing systems and organ conformable panels, some including one or more passive ultrasound detectable markers and some omitting such markers. Method 100 may likewise be carried out with similar ultrasound sensing systems and organ conformable panels.

As indicated by block 2104, an organ conformable panel is positioned on an exterior surface of an internal organ. The organ conformable panel is acoustically distinguishable from the internal organ and identifying the portion of the internal organ for removal. The panel may be acoustically distinguishable from the internal organ by having and acoustic impedance different than that of the tissue forming the internal organ. The organ conformable panel may identify the portion of the internal organ designated for removal with a passive ultrasound detectable marker or a plurality of such markers supported by the organ conformable panel. In some implementations, the passive ultrasound detectable marker or markers may form an array or grid for mapping the organ surface, wherein the resulting map is used to identify those portions of the underlying organ designated for removal.

In some implementations, the organ conformable panel may identify the portion of the internal organ designated for removal with an internal edge extending about an aperture position over the tumor or organ portion designated for removal or may identify the portion of the internal organ designated for removal with an outer peripheral edge of the panel, wherein the panel is sized and shaped to correspond to the portion of the organ designated for removal and is aligned with and positioned on top of the portion of the organ designated for removal.

As indicated by block 2106, an organ surface ultrasound probe is positioned on an exterior surface of the organ. The organ surface ultrasound probe is positioned such that its field-of-view encompasses the passive ultrasound detectable marker (or markers) while on the exterior surface of the internal organ. In some implementations, the probe is positioned such to capture the organ conformable panel supports the passive ultrasound detectable markers.

As indicated by block 2108, the organ surface ultrasound probe is used to acoustically detecting the organ conformable panel. In particular, the organ surface ultrasound probe may emit ultrasound waves or energy and then sense those ultrasound waves are energy that have been reflected (the echo) from the organ conformable panel (and any passive ultrasound detectable markers, when provided). The ultrasound data acquired by the organ surface ultrasound probe may be used to identify and locate the portion of the internal organ designated for removal relative to a cutting tool used during the medical procedure.

As indicated by block 2110, the cutting tool is used to remove the portion of the internal organ based upon the data acquired from the acoustic detecting of the organ conformable panel by the organ surface ultrasound probe. As discussed above, in some implementations, the cutting tool may cut through the organ conformable panel and cut into the underlying organ to remove the identified and located portions of the organ. As discussed above, in some implementations, the cutting tool may move along an outer edge or an internal edge of the organ conformable panel when removing the identified and located portions of the organ.

Although the claims of the present disclosure are generally directed to particular ultrasound sensing systems, the present disclosure is additionally directed to the features set forth in the following definitions.
1. An ultrasound sensing system comprising:
   an organ conformable panel to be positioned on an exterior surface of an internal organ, the organ conformable panel having a first acoustic impedance; and
   a passive ultrasound detectable marker supported by the organ conformable panel, the marker having a second acoustic impedance different than the first acoustic impedance.
2. The ultrasound sensing system of definition 1, wherein the passive ultrasound detectable marker is part of a two-dimensional array of passive ultrasound detectable markers supported by the organ conformable panel, each of the passive ultrasound detectable markers having an acoustic impedance different than the first acoustic impedance.
3. The ultrasound sensing system of definition 1 further comprising a second passive ultrasound detectable marker supported by the organ conformable panel, the second marker having a third acoustic impedance different than the first acoustic impedance and different than the second acoustic impedance.
4. The ultrasound sensing system of definition 1 further comprising an adhesive layer on a first face of the organ conformable panel to adhere the organ conformable panel to the exterior surface of the internal organ.
5. The ultrasound sensing system of definition 4, wherein the passive ultrasound detectable marker is optically visible from a second face of the organ conformable panel.
6. The ultrasound sensing system of definition 4, wherein the passive ultrasound detectable marker is not optically visible from a second face of the organ conformable panel and wherein the ultrasound sensing system further comprises an optical marker on the second face of the organ conformable panel, the optical marker having a location corresponding to the location of the passive ultrasound detectable marker.
7. The ultrasound sensing system of definition 1, wherein the organ conformable panel has a first face to face the exterior surface of the organ and a second face opposite the first face and wherein the passive ultrasound detectable marker is optically visible on the second face.
8. The ultrasound sensing system of definition 1, wherein the organ conformable panel has a first face to face the exterior surface of the organ and a second face opposite the first face and wherein the passive ultrasound detectable marker is not optically visible the second face, the ultrasound sensing system further comprising an optical marker supported by the organ conformable panel at a location corresponding to the passive ultrasound detectable marker, the optical marker being optically visible from the second face.
9. The ultrasound sensing system of definition 1, wherein the organ conformable panel has a first face to face the exterior surface of the organ in a second face opposite the first face and wherein the passive ultrasound detectable marker is part of a two-dimensional array of passive ultrasound detectable markers, wherein each of the passive ultrasound detectable markers has an acoustic impedance different than the first acoustic impedance and is optically visible from the second face.
10. The ultrasound sensing system of definition 1, wherein the organ conformable panel has a first face to face the exterior surface of the organ and a second face opposite the first face and wherein the passive ultrasound detectable marker is part of a two-dimensional array of passive ultrasound detectable markers, wherein each of the passive ultrasound detectable markers has an acoustic impedance different than the first acoustic impedance and is not optically visible from the second face, the ultrasound sensing system further comprising a two-dimensional array of optical markers optically visible from the second face, the optical markers of the two-dimensional array being supported by the organ conformable panel at locations corresponding to locations of the passive ultrasound detectable markers.
11. The ultrasound sensing system of definition 1, wherein the organ conformable panel comprises a gel.
12. The ultrasound sensing system of definition 1, wherein the organ conformable panel comprises a fabric.
13. The ultrasound sensing system of definition 1, wherein the passive ultrasound detectable marker is bonded to the organ conformable panel.
14. The ultrasound sensing system of definition 1, wherein the passive ultrasound detectable marker is integrally formed as part of a single unitary body with the organ conformable panel.
15. The ultrasound sensing system of definition 1, wherein the passive ultrasound detectable marker is sewn into the organ conformable panel.
16. The ultrasound sensing system of definition 1, wherein the passive ultrasound detectable marker is coated upon the organ conformable panel.
17. The ultrasound sensing system of definition 1, wherein the passive ultrasound detectable marker is shaped and located to identify a targeted region of the organ for surgical removal.
18. The ultrasound sensing system of definition 1, wherein the passive ultrasound detectable marker is shaped and located to identify portions of the organ not designated for surgical removal.
19. The ultrasound sensing system of definition 1, wherein the organ conformable panel is sufficiently flexible to bend with bending of the organ.
20. The ultrasound sensing system of definition 1, wherein the organ conformable panel is stretchable.
21. The ultrasound sensing system of definition 1, wherein the organ conformable panel is sufficiently flexible so as to overlie and conform to a tumor bulging from the exterior surface of the organ.
22. The ultrasound sensing system of definition 21, wherein the tumor is designated for removal by a cutting tool and wherein the organ conformable panel is cuttable by the cutting tool.
23. The ultrasound sensing system of definition 1, further comprising an organ surface ultrasound probe to be positioned on the organ to detect the passive ultrasound detectable marker.
24. The ultrasound sensing system of definition 23, wherein the organ conformable panel comprises an optically detectable marker at a location corresponding to the passive ultrasound detectable marker, the system further comprising an optical sensor to be focused on optically detectable marker panel.
25. The ultrasound sensing system of definition 24 further comprising a controller to register detected locations of the optically detectable marker and the passive ultrasound detectable marker.
26. A method comprising:
   providing a passive ultrasound detectable marker on an exterior surface of an internal organ;
   positioning an organ surface ultrasound probe on the exterior surface of the internal organ; and
   acoustically detecting a position of the passive ultrasound detectable marker with the organ surface ultrasound probe.
27. The method of definition 26 further comprising:
   positioning an optical camera proximate the exterior surface of the internal organ; and
   optically imaging the passive ultrasound detectable marker with the optical camera.
28. The method of definition 27 further comprising registering a first location of the passive ultrasound detectable marker as acoustically detected by the organ surface ultrasound probe with a second location of the passive ultrasound detectable marker as optically imaged by the optical camera.
29. The method of definition 28 further comprising cutting a portion of the internal organ based upon at least one of the first location and the second location.
30. The method of definition 26 further comprising cutting a portion of the internal organ while the passive ultrasound detectable marker is on the exterior surface of the internal organ and based upon the acoustically detected position of the passive ultrasound detectable marker.
31. The method of definition 26, wherein the passive ultrasound detectable marker is coated directly onto the exterior surface of the organ.
32. The method of definition 31, wherein the passive ultrasound detectable marker is sprayed onto the exterior surface of the organ.
33. The method of definition 26, wherein the passive ultrasound detectable marker is supported by an organ conformable panel and wherein the passive ultrasound detectable marker is provided on the exterior surface of the internal organ by positioning the organ conformable panel on the exterior surface of the internal organ.
34. The method of definition 33 further comprising cutting and removing a portion of the internal organ while the organ conformable panel is positioned on the exterior surface of the internal organ.
35. The method of definition 34 further comprising cutting a portion of the organ conformable panel while cutting and removing a portion of the internal organ.
36. The method of definition 33, wherein an optically detectable marker is supported by the organ conformable panel at a first location corresponding to a second location of the passive ultrasound detectable marker, the method further comprising:
   positioning an optical camera proximate the exterior surface of the internal organ; and
   optically imaging the optically detectable marker with the optical camera.
37. The method of definition 36 further comprising:
   registering the first location and the second location; and
   cutting and removing a portion of the internal organ based upon the registered first location and second location.
38. The method of definition 26, wherein the passive ultrasound detectable marker indicates a location of a portion of the internal organ for surgical removal, the method further comprising surgically removing the portion of the internal organ based upon the acoustically detected position of the passive ultrasound detectable marker.
39. The method of definition 26, wherein the passive ultrasound detectable marker is part of a two-dimensional array of passive ultrasound detectable markers on the exterior surface of the organ.
40. The method of definition 39, wherein the two-dimensional array of passive ultrasound detectable markers are supported by an organ conformable panel, the method further comprising positioning the organ conformable panel on the exterior surface of the organ.
41. The method of definition 40, wherein the internal organ comprises a tumor designated for removal and wherein the method further comprises positioning the organ conformable panel and the two-dimensional array of passive ultrasound detectable markers on top of the tumor.
42. The method of definition 41, wherein the organ conformable panel is positioned such that portions of the organ conformable panel and some of the passive ultrasound detectable markers of the two-dimensional array outwardly extend beyond the tumor on the exterior surface of the internal organ.
43. A method comprising:
   positioning an organ conformable panel on an exterior surface of an internal organ, the organ conformable panel being acoustically distinguishable from the internal organ and identifying a portion of the internal organ for removal;
   positioning an organ surface ultrasound probe on the exterior surface of the organ;
   acoustically detecting the organ conformable panel with the organ surface ultrasound probe; and
   removing the portion of the internal organ based upon data acquired from the acoustic detecting of the organ conformable panel.
44. The method of definition 43. wherein the organ conformable panel has a first acoustic impedance and supports a passive ultrasound detectable marker having a second acoustic impedance different than the first acoustic impedance, the passive ultrasound detectable marker identifying the portion of the internal organ for removal.
45. The method of definition 43, wherein the organ conformable panel has an edge identifying the portion of the internal organ for removal.
46. The method of definition 45, wherein the organ conformable panel has an opening, the edge extending along the opening.
47. The method of definition 45, wherein the edge is an outer peripheral edge of the organ conformable panel.
48. The method of definition 43 further comprising optically determining a positioning of the organ conformable panel on the exterior surface of the organ while acoustically detecting the organ conformable panel with the organ surface ultrasound probe.
49. The method of definition 48, wherein the organ conformable panel supports a passive ultrasound detectable marker, the method further comprising optically detecting a position of the passive ultrasound detectable marker.
50. The method of definition 49, wherein the optical detecting of the position of the passive ultrasound detectable marker comprise optically imaging the passive ultrasound detectable marker while the organ conformable panel is on the internal organ.
51. The method of definition 49, wherein the organ conformable panel supports a passive ultrasound detectable marker which is not visible when the organ conformable panel is on the internal organ and wherein the organ conformable panel comprises an optical marker at a location corresponding to the position of the passive ultrasound detectable marker, wherein the optical detecting of the position of the passive ultrasound detectable marker comprise optically imaging the optical marker while the organ conformable panel is on the internal organ.
52. The method of definition 43 further comprising forming a passive ultrasound detectable marker on the organ conformable panel while the organ conformable panel is on the exterior surface of the internal organ.
53. The method of definition 52, wherein the forming of the passive ultrasound detectable marker on the organ conformable panel comprises coating the passive ultrasound detectable marker on the organ conformable panel while the organ conformable panel is on the exterior surface of the internal organ.
54. An apparatus comprising:
   an organ conformable panel having a face to extend across an external surface of an organ, the face comprising:
      a first portion formed from a first material having a first acoustic impedance; and
      a second portion formed from a second material having a second acoustic impedance different than the first acoustic impedance.

Although the present disclosure has been described with reference to example implementations, workers skilled in the art will recognize that changes may be made in form and detail without departing from the disclosure. For example, although different example implementations may have been described as including features providing various benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following definitions is manifestly intended to be as broad as possible. For example, unless specifically otherwise noted, the definitions reciting a single particular element also encompass a plurality of such particular elements. The terms “first”, “second”, “third” and so on in the definitions merely distinguish different elements and, unless otherwise stated, are not to be specifically associated with a particular order or particular numbering of elements in the disclosure.

What is claimed is:

1. An ultrasound sensing system comprising:
an organ conformable panel to be positioned on an exterior surface of an internal organ, the organ conformable panel having a first acoustic impedance; and
a passive ultrasound detectable marker supported by the organ conformable panel, the marker having a second acoustic impedance different than the first acoustic impedance.

2. The ultrasound sensing system of claim 1, wherein the passive ultrasound detectable marker is part of a two-dimensional array of passive ultrasound detectable markers supported by the organ conformable panel, each of the passive ultrasound detectable markers having an acoustic impedance different than the first acoustic impedance.

3. The ultrasound sensing system of claim 1 further comprising a second passive ultrasound detectable marker supported by the organ conformable panel, the second marker having a third acoustic impedance different than the first acoustic impedance and different than the second acoustic impedance.

4. The ultrasound sensing system of claim 1 further comprising an adhesive layer on a first face of the organ conformable panel to adhere the organ conformable panel to the exterior surface of the internal organ.

5. The ultrasound sensing system of claim 4, wherein the passive ultrasound detectable marker is optically visible from a second face of the organ conformable panel.

6. The ultrasound sensing system of claim 1, wherein the organ conformable panel has a first face to face the exterior surface of the organ and a second face opposite the first face and wherein the passive ultrasound detectable marker is optically visible on the second face.

7. The ultrasound sensing system of claim 1, wherein the organ conformable panel has a first face to face the exterior surface of the organ and a second face opposite the first face and wherein the passive ultrasound detectable marker is not optically visible the second face, the ultrasound sensing system further comprising an optical marker supported by the organ conformable panel at a location corresponding to the passive ultrasound detectable marker, the optical marker being optically visible from the second face.

8. The ultrasound sensing system of claim 1, wherein the organ conformable panel comprises a gel.

9. The ultrasound sensing system of claim 1, wherein the organ conformable panel comprises a fabric.

10. The ultrasound sensing system of claim 1, wherein the passive ultrasound detectable marker is shaped and located to identify a targeted region of the organ for surgical removal.

11. The ultrasound sensing system of claim 1, wherein the passive ultrasound detectable marker is shaped and located to identify portions of the organ not designated for surgical removal.

12. The ultrasound sensing system of claim 1, wherein the organ conformable panel is sufficiently flexible so as to overlie and conform to a tumor bulging from the exterior surface of the organ.

13. The ultrasound sensing system of claim 12, wherein the tumor is designated for removal by a cutting tool and wherein the organ conformable panel is cuttable by the cutting tool.

14. A method comprising:
providing a passive ultrasound detectable marker on an exterior surface of an internal organ;
positioning an organ surface ultrasound probe on the exterior surface of the internal organ; and
acoustically detecting a position of the passive ultrasound detectable marker with the organ surface ultrasound probe.

15. The method of claim 14 further comprising:
positioning an optical camera proximate the exterior surface of the internal organ; and
optically imaging the passive ultrasound detectable marker with the optical camera.

16. The method of claim 14 further comprising cutting a portion of the internal organ while the passive ultrasound detectable marker is on the exterior surface of the internal organ and based upon the acoustically detected position of the passive ultrasound detectable marker.

17. The method of claim 14. wherein the passive ultrasound detectable marker is coated directly onto the exterior surface of the organ.

18. The method of claim 14, wherein the passive ultrasound detectable marker is supported by an organ conformable panel and wherein the passive ultrasound detectable marker is provided on the exterior surface of the internal organ by positioning the organ conformable panel on the exterior surface of the internal organ.

19. The method of claim 14, wherein the passive ultrasound detectable marker is part of a two-dimensional array of passive ultrasound detectable markers on the exterior surface of the organ.

20. An apparatus comprising:
an organ conformable panel having a face to extend across an external surface of an organ, the face comprising:
a first portion formed from a first material having a first acoustic impedance; and
a second portion formed from a second material having a second acoustic impedance different than the first acoustic impedance.
